(19) United States
(12) Patent Application Publication
(10) Pub. No.: US 2021/0382040 A1
(43) Pub. Date: Dec. 9, 2021

(54) DETECTION OF BIOLOGICALS PENETRATION INTO TISSUE SURROGATES

(71) Applicant: VISIKOL, INC., Whitehouse Station, NJ (US)

(72) Inventors: Thomas Villani, Monmouth Junction, NJ (US); Nicholas Crider, North Landing, IA (US); Michael Johnson, Bridgewater, NJ (US); Joseph Napoli, Branchburg, NJ (US)

(73) Assignee: VISIKOL, INC., Whitehouse Station, NJ (US)

(21) Appl. No.: 17/288,060

(22) PCT Filed: Mar. 26, 2018
(86) PCT No.: PCT/US2018/052796

§ 371 Date: Apr. 23, 2021

(51) Int. Cl. G01N 33/533; G01N 1/30; G02B 21/00; G01N 33/563

(52) U.S. Cl. CPC G01N 33/533; G01N 33/563; G02B 21/0076; G01N 1/30

(57) ABSTRACT

Provided are methods of assessing penetration of biologicals into surrogate tissues.

### FIELD OF THE INVENTION

The present disclosure relates to histology and human disorder diagnostics. More specifically, this disclosure relates to in vitro assessment of the distribution and penetration of therapeutic biologicals into tissue surrogates.

### BACKGROUND

Therapeutic strategies employing therapeutic and/or diagnostic substances have offered great promise in the treatment of cancer by enabling or enhancing malignant cell destruction. Of recent success, checkpoint inhibitor antibodies, such as those directed against cytotoxic T lymphocyte antigen-4 (CTLA-4) and programmed death-1 (PD-1) have been shown to disrupt interactions that prevent T-cell activation and thus facilitate immune destruction of cancerous cells. Other antibodies such as those targeting growth factors and integrins have been successfully employed to inhibit angiogenesis in the tumor microenvironment, thus limiting cancer cell survival and proliferation. Moreover, through conjugation or particle formulation, human antibodies have been leveraged to deliver payloads of drugs or radioisotopes thus facilitating efficient, specific destruction of cancerous cells while limiting off target effects.

However, in the context of treating solid tumor malignancies, the delivery of such therapeutic substances may be difficult, since intratumoral transport of therapeutic agents can be impaired by the densely-packed extracellular matrix known to exist in the tumor microenvironment. Accordingly, systemic clearance of these therapeutic substances can outpace tumor penetration, thus limiting therapeutic efficacy. Various approaches including co-administration of biological therapeutics with tumor-penetrating peptides or modulation of biological affinity, size, and dose have been used to enhance penetration into tumors In order to interrogate the effects of these strategies, rapid, reliable quantification methods to assess the ability of new therapeutics to deeply penetrate tumors is needed.

Antibody penetration is used in in vivo models, presents a number of drawbacks for application in the high content screening of therapeutic candidates. For example, the two-dimensional (2D) nature of tumor isolation, sectioning and immunofluorescence staining required for assessment of antibody penetration depth in these assays limits spatial interpretation of antibody distribution within the tumor. Serial sectioning and stereological approaches have sought to reconstruct such data for a three-dimensional (3D) interpretation, but these approaches are time consuming, expensive, and fail to fully recapitulate 3D penetration. Chemical clearing of excised tumors combined with 3D fluorescence microscopy has been used for the visualization of antibody penetration in 3D. However, the rapid, iterative-nature of antibody-based therapeutic development necessitates a method for assaying antibody penetration in a faster, more inexpensive manner than such in vivo studies can facilitate.

While 3D tumor cell cultures have previously been utilized as models to study therapeutic antibody penetration, current methods fail to rapidly and accurately quantify penetration depth for a number of reasons. For example, radiolabeled antibody incubation coupled with cryosectioning and autoradiography has resulted in visualization of antibody penetration in tumor 3D cell cultures, regardless of bound versus un-bound state. Also, using confocal microscopy, antibody penetration in 3D tumor cell cultures has been visualized, but optical attenuation of signal past a few cell layers in the 3D cell culture is interpreted as positive, despite the fact that only the exterior cells will be characterized where penetration is the most uniform and complete. Moreover, quantification methods based on confocal microscopy visualization of antibody penetration are limited to analysis of spherical penetration where fluorescence intensity is measured over increasingly larger circular areas from confocal image slices to determine antibody penetration depth. However, since tumor 3D cell cultures are often non-symmetrical or irregular in shape, this approach fails to produce accurate quantification of antibody penetration depth.

Therefore, there is a need for better methodologies to assess the penetration depth of antibodies into 3D tumor cell cultures in drug development contexts

### SUMMARY

It has been discovered that the ability of biologicals, such as antibodies, to penetrate into tissue model surrogates, such as 3D cell cultures and solid tumor models, can be quantified through the combination of confocal microscopy, tissue clearing, fluorescent labeling and a computer program that measures the gradients of concentrations of such biologicals throughout the tissue surrogate based on analysis of the profile gradients of intensity of pixel value along multiple paths in the resultant confocal images.

These discoveries have been exploited to provide the present disclosure, which, in part, provides a method of determining the depth a biological can penetrate into a tissue surrogate. The method comprises: treating the tissue surrogate with the biological, or binding fragment thereof, which binds specifically to a predetermined target in and/or on the tissue surrogate; contacting the biological-treated tissue surrogate with a labeled binding agent specific for the biological or fragment thereof; imaging the bound label in the microtissue with a 3-D optical microscope and generating an image stack; calculating the amount and location of biological penetration into the tissue surrogate by reading the image stack into memory of a digital image processor and processing the image within the digital image processor and the memory, the image being processed by: separating the image stack into substacks by wavelength, locating the tissue surrogate in the image stack, identifying a center plane of a tissue surrogate in the image stack, identifying a center of the tissue surrogate, and measuring an intensity of the bound label at a plurality of distances from the center of the tissue surrogate; and the image further being processed by performing a statistical analysis of the measured intensity of the bound label to determine a measure of the depth a biological can penetrate into the tissue surrogate.

In certain embodiments, the tissue surrogate is a portion of a cultured tissue. In certain embodiments, the tissue surrogate is fixed with a chemical fixative before it is treated with the biological.

In some embodiments, the clearing agent comprises HISTO-M water, Focusclear, BABB, 3DISCO, iDISCO, CUBIC, PACT, PARS, CLARITY, Scale, ClearT, glycerol ClearT2, uDISCO or SeeDB.

In certain embodiments, the 3-D microscope is a confocal, two-photon, or light-sheet microscope.

In some embodiments the biological is an antibody or binding fragment thereof. In certain embodiments, the antibody is a monoclonal antibody, antibody mimetic, camelid, humanized antibody, chimeric antibody, or binding fragment thereof. In specific embodiments, the binding fragment of the antibody is an F(ab), Fab′, F(ab′)2, Fv, sFv, r IgG, Fab-H, IgG typeM, Fc5-H, or Fc.

In some embodiments, the binding agent is a receptor, a protein that binds to a receptor on/in the tissue surrogate, an antibody, or binding fragment thereof.

In certain embodiments, the binding agent is labeled, and in specific embodiments, the label is a fluorescent or colorimetric molecule. In some embodiments, the location and concentration of biological that has bound to the tissue surrogate is determined by measuring the intensity of the fluorescent label bound thereto. In specific embodiments, the fluorescence intensity is measured across multiple arbitrary linear paths within a z-slice. In some embodiments, the intensity is measured by measuring bandwidth. In certain embodiments, the fluorescent intensity is calculated with a modified image J program.

In some embodiments, the measure of the depth a biological can penetrate into the tissue surrogate is determined from measuring the bandwidth or intensity value of a plurality of pixels comprising the image obtained from the tissue surrogate. The program calculates the “bandwidth” defined as the distance between pixels at half the maximal pixel value which span the maximum point, e.g, the program finds the “shoulders” of the peak and measures the distance between them. This value represents the spread of the antibody concentration along a specified path.

In another aspect, the disclosure provides a method of determining the depth a biological can penetrate into a tissue surrogate. The method comprises: treating the tissue surrogate with a labeled biological, or binding fragment thereof, which binds specifically to a predetermined target in and/or on the tissue surrogate; imaging the bound label in the tissue surrogate with a 3-D optical microscope and generating an image stack; calculating the amount and location of biological penetration into the tissue surrogate by reading the image stack into memory of a digital image processor and by processing the image within the digital image processor and the memory, the image being processed by: separating the image stack into substacks by wavelength; locating the microtissue in the image stack; identifying a center plane of a microtissue in the image stack; identifying a center of the tissue surrogate; measuring an intensity of the bound label at a plurality of distances from the center of the tissue surrogate; and performing statistical analysis of the measured intensity of the bound label to determine a measurement of the depth a biological can penetrate into the tissue surrogate.

In some embodiments, the tissue surrogate is fixed with a chemical fixative before it is treated with the biological.

In some embodiments, the clearing agent comprises HistoM, water, Focusclear, BABB, 3DISCO, iDISCO, CUBIC, PACT, PARS, CLARITY, Scale, ClearT, glycerol ClearT2, uDISCO or SeeDB.

In some embodiments, the 3-D microscope is a confocal, two-photon, or light-sheet microscope.

In certain embodiments, the biological is an antibody, or binding fragment thereof. In certain embodiments, the antibody is a monoclonal antibody, antibody mimetic, camelid, humanized antibody, chimeric antibody, or binding fragment thereof. In specific embodiments, the binding fragment of the antibody is an F(ab), Fab′, F(ab′)2, Fv, sFv, r IgG, Fab-H, IgG typeM, Fc5-H, or Fc.

In some embodiments, the label is a fluorescent or colorimetric molecule.

In particular embodiments, the location and concentration of the biological that has bound to the microtissue is determined by measuring the intensity of the fluorescent label bound thereto. In certain embodiments, the fluorescence intensity is measured across multiple user-inputted linear paths within a z-slice. In particular embodiments, the fluorescent intensity is calculated with a modified image J program.

### BRIEF DESCRIPTION OF THE DRAWINGS

The foregoing and other objects of the present disclosure, the various features thereof, as well as the invention itself may be more fully understood from the following description, when read together with the accompanying drawings, in which:

FIG. 1 is a flow chart showing the calculation process for antibody penetration method according to the disclosure.

FIG. 2 is a graphic representation of Dₘₐₓ, Vₘₐₓ, and bandwidth values on a representative antibody concentration profile curve;

FIG. 3A is a graphic representation of the intensity profiles for A498 spheroids without any treatment (control) wherein “Pt-#” refers to particular points selected in the image, representing end-points to measure intensity profiles along from the center point (Pt-1);

FIG. 3B is a representation of a confocal micrograph of untreated A498 spheroids stained with IgG-AlexaFluor488 conjugate;

FIG. 4A is a graphic representation of intensity profiles (pixel intensity) for A498 spheroids treated for 10 minutes with mouse anti-human human β-integrin antibodies;

FIG. 4B is a confocal micrograph of A498 spheroids treated for 10 minutes with anti-human β-integrin antibody and copy stained with IgG-AlexaFluor488 conjugate;

FIG. 5A is a graphic confocal representation of intensity profiles for A498 spheroids treated for 4 hr. with mouse anti-human human β-integrin antibodies;

FIG. 5B is a representation of a confocal micrograph of A498 spheroids treated for 4 hr. with mouse anti-human human β-integrin antibodies and stained with IgG-AlexaFluor488 conjugate;

FIG. 6A is a graphic representation showing the Vₘₐₓ of pixel intensity for A498 spheroids treated with a mouse anti-human β-integrin antibody for 0 min (control), 10 min, or 4 hours, where the paths shown on the x axis corresponds to the path between the center point and the points encircling the 3D cell culture model on the periphery of FIGS. 2B, 3B, and 4B;

FIG. 6B is a graphic representation showing the Vₘₐₓ (microns) for A498 spheroids treated with mouse anti-human β-integrin antibody for 0 minutes (control), 10 minutes, or 4 hours;

FIG. 7 is a graphic representation showing the AUC (microns) for A498 spheroids treated with a mouse anti-human β-integrin antibody for 0 minutes (control), 10 minutes, or 4 hours;

FIG. 8A is a graphic representation showing the Average Vₘₐₓ (pixel intensity) for A498 spheroids treated with a β-integrin antibody for 0 minutes (control), 10 minutes, or 4 hours;

FIG. 8B is a graphic representation showing the Average Dₘₐₓ (microns) for A498 spheroids treated with a mouse anti-human β-integrin antibody for 0 minutes (control), 10 minutes, or 4 hours;

FIG. 8C is a graphic representation showing the Average AUC (microns) for A498 spheroids treated with a mouse anti-human β-integrin antibody for 0 minutes (control), 10 minutes, or 4 hours;

FIG. 9A is a graphic representation showing the intensity profile for HCT116 spheroids not treated with a mouse anti-human β-integrin antibody;

FIG. 9B is a confocal micrograph of HCT116 spheroids stained with mouse ant-human β-integrin antibody and stained with IgG-AlexaFluor488 conjugate;

FIG. 10A is a graphic representation showing the intensity profile (pixels) for HCT116 spheroids treated with a mouse anti-human β-integrin antibody for 10 minutes;

FIG. 10B is a confocal micrograph of HCT116 spheroids treated with a β-integrin antibody for 10 minutes;

FIG. 11A is a graphic representation showing the intensity profile (pixels) for HCT116 spheroids treated with a mouse anti-human β-integrin antibody for 4 hours;

FIG. 11B is a confocal micrograph of HCT116 spheroids treated with a mouse anti-human β-integrin antibody for 4 hours;

FIG. 12A is a graphic representation showing the Vₘₐₓ (pixel intensity) for HCT116 spheroids treated with mouse-anti-human β-integrin antibody for 0 minutes (control), 10 minutes, or 4 hours;

FIG. 12B is a graphic representation showing the Average Vₘₐₓ (pixel intensity) for HCT116 spheroids treated with mouse-anti-human β-integrin antibody for 0 minutes (control), 10 minutes, or 4 hours;

FIG. 13A is a graphic representation showing the Dₘₐₓ (microns) for HCT116 spheroids treated with mouse-anti-human β-integrin antibody for 0 minutes (control), 10 minutes, or 4 hours;

FIG. 13B is a graphic representation showing the Average Dₘₐₓ (microns) for HCT116 spheroids treated with mouse-anti-human β-integrin antibody for 0 minutes (control), 10 minutes, or 4 hours;

FIG. 14A is a graphic representation showing the AUC (microns*pixel intensity) for HCT116 spheroids treated with mouse-anti-human β-integrin antibody for 0 minutes (control), 10 minutes, or 4 hours;

FIG. 14B is a graphic representation showing the Average AUC (microns*pixel intensity) for HCT116 spheroids treated with mouse-anti-human β-integrin antibody for 0 minutes (control), 10 minutes, or 4 hours;

FIG. 15A is a graphic representation showing the bandwidth (microns) for each profile of paths for A498 spheroids treated with mouse-anti-human β-integrin antibody for 10 minutes and 4 hours;

FIG. 15B is a graphic representation showing the bandwidth (microns) for each profile of paths for HCT116 spheroids treated with mouse-anti-human β-integrin antibody for 10 minutes and 4 hours; and

FIG. 16 is a diagrammatic representation of the calculation process using the computer program.

### DESCRIPTION

The disclosures of any patents, patent applications, and publications referred to herein are hereby incorporated by reference in their entireties into this application in order to more fully describe the state of the art known to those skilled therein as of the date of the invention described and claimed herein.

Unless defined otherwise, all technical terms used herein have the same meaning as commonly understood by one of ordinary skill in the art to which this disclosure belongs. The initial definition provided for a group or terms herein applies to that group or term throughout the present specification individually or as part of another group, unless otherwise indicated.

As used herein, a “3D cell culture” encompasses all cell culture models that are more than one cell layer in thickness. This includes, but is not limited to, the colloquial terms spheroid, microtissue and organoid. These models can be a single cell type, multiple cell types and range in size from a few cells to several millimeters in thickness.

The term “biologicals” as used here encompasses therapeutic and diagnostic substances derived from biological sources, such as, but not limited to, proteins, enzymes, or and antibodies used to locate, identify, and/or treat a disorder or disease.

As used herein, a “tissue surrogate” encompasses any ex vivo or in vitro model which recapitulates any features of in vivo tissues. This includes but is not limited to 3D cell cultures, cultured xenografts (tissue pieces removed from patient or animal and maintained alive for study), patient derived xenografts (tissues resected from patient and cultured), matrices impregnated with more than one layer of cells, 3D-printed tissues, precision cut lung slices, pancreatic islets. These models can be a single cell type, multiple cell types, and range in size from a few cells to several millimeters in thickness. In contrast to traditional 2D cell cultures which are adherent cells on a plate, tissue surrogates from patient-derived xenografts (PDX) facilitate the production of a 3D tumor microenvironment that is a more physiologically relevant obstacle to intratumoral antibody penetration.

The present disclosure provides a novel method of evaluating the relative propensity of various therapeutic and/or diagnostic biologicals to penetrate tissue surrogates. The method employs fluorescent or radio-labeling labeling and 3D microscopy to detect and quantify the depth of penetration of biologicals into tissue surrogates. This is combined with analysis of label intensities across arbitrary linear paths within z-stacks to enable rapid detection and quantification of the penetration of a biological into the surrogate tissue, regardless of shape irregularities.

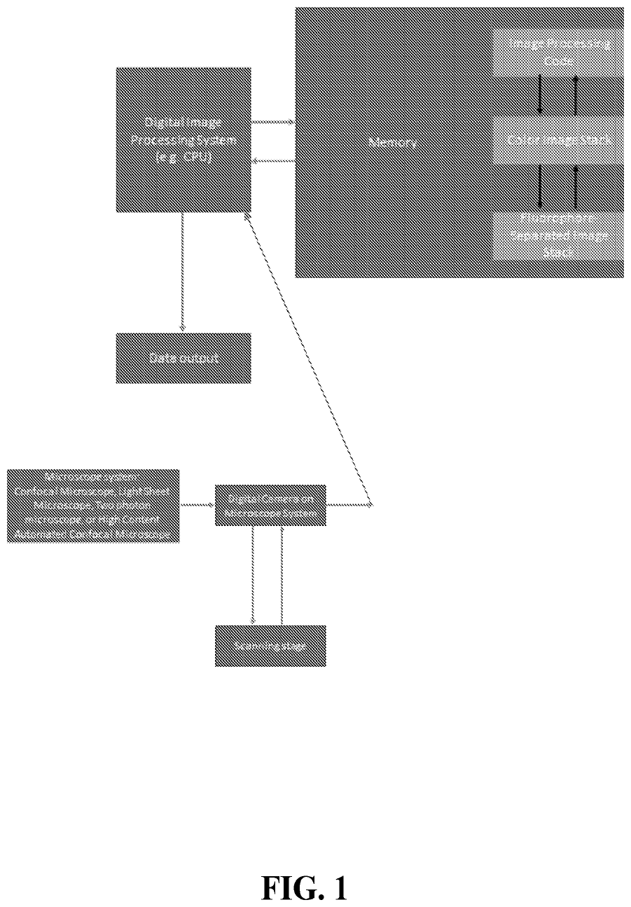

FIG. 1 depicts the method according to the disclosure. The method comprises: treating the tissue surrogate with the biological, or binding fragment thereof, which binds specifically to a predetermined target in and/or on the tissue surrogate. The biological-bound tissue surrogate is then contacted with a labeled binding agent specific for the biological or fragment thereof. The bound label then then imaged with a 3-D optical microscope and an image stack is generated. The amount and location of biological penetration into the tissue surrogate is calculated by reading the image stack into memory of a digital image processor and processing the image within the digital image processor and the memory. The image is processed by: separating the image stack into substacks by wavelength, locating the tissue surrogate in the image stack, identifying a center plane of a tissue surrogate in the image stack, identifying a center of the tissue surrogate, and measuring an intensity of the bound label at a plurality of distances from the center of the tissue surrogate; and the image further being processed by performing a statistical analysis of the measured intensity of the bound label to determine a measure of the depth a biological can penetrate into the tissue surrogate.

The tissue surrogates used in the present method are tissue fragments or cells which have been cultured into tissue cultures that are more than one cell layer in thickness. These tissue surrogates are e.g., useful as in vitro and ex vivo models for various therapeutic applications such as, but not limited to, cancer, drug-induced liver injury, epilepsy, lung disease, and diabetes. Thus, tissue surrogate models can be used to determine treatment options for such disorders. Tissue surrogates are also useful in diagnostic assays such as one which enables the determination of the depth a particular biological drug can penetrate into a tissue, as described herein.

Tissue surrogates can be generated by resection of cells or tissue pieces from animals or humans and cultured in vitro using standard culturing techniques. Resected pieces may be treated to remove cells, biochemicals, extracellular matrix, or other constituents prior to culturing. Alternatively, tissue surrogates can be commercially obtained e.g., from Biopta (Beltsville, Md.), Creative BioArray (Shirley, N.Y.), and Charles River Laboratories (Stone Ridge, N.Y.). Tissue surrogates also include 3D cell culture models, as described herein.

The 3D cell cultures used in the present method are tissue fragments or cells which have been cultured into a 3D tissue cultures that are more than one cell layer in thickness. These 3D cell cultures are useful as in vitro models for various therapeutic applications such as, but not limited to, cancer, drug induced liver injury, epilepsy, and diabetes. Thus, 3D cell culture models can be used to determine treatment options for such disorders. 3D cell cultures are also useful in diagnostic assays such as one which enables the determination of the depth a particular biological can penetrate into a tissue, as described herein.

3D cell cultures can be generated from cell lines such as, but not limited to, cancer cell lines (e.g., A498 and HTC116), whole tissues, stem cells and iPSC cells using ultra-low-attachment well plates, the hanging drop approach, scaffolds, bio-printing or several other techniques. Alternatively, 3D cell cultures can be commercially obtained e.g., from InSphero (Schlieren, SU), Stemonix (Maple Grove, Minn.) and Tara Biosystems (New York, N.Y.).

Before being treated with a biological, the tissue surrogate may or may not be fixed. Useful fixatives include, but are not limited to formaldehyde, glutaraldehyde, acetone, methanol, ethanol, paraformaldehyde, HOPE fixative, osmium tetroxide, Davidson's Fixative, Bouin's Fixative, buffered formalin. These are commercially available from, e.g., Fisher Scientific (Waltham, Mass.). For example, the microtissues are place in 10% neutral buffered formalin, for 10 minutes at room temperature (RT).

The tissue surrogates are contacted with a biological drug, including but not limited to, an intact or fragmented antibody, protein, peptide, enzyme, protein-fragment, RNA or RNA-fragment, DNA or DNA-fragment, hormone, glycoside, glycopeptide, toxin, or other biomolecule intended to effect or modulate the biology and/or biochemistry of disease, which binds to a specific site within the tissue surrogate. These biologicals may be obtained e.g., from BioChemed Services (Winchester, Va.). The biological drug may also be a virus modified or intended to deliver a gene into the tissue surrogate upon treatment, and may be obtained e.g., from Vigenebio (Rockville, Md.).

As used herein, the term “antibody” encompasses any type of antibody, (IgG, IgM, IgD, IgE, IgA, etc.) from any species (mouse, human, bovine, camel, chicken, rat, guinea pig, etc.). It may also be a monoclonal antibody, humanized antibody, chimeric antibody, camelid, antibody mimetic, affibody, or binding fragment of such antibodies, including but not limited to an sFv, Fab, F(ab′)2, Fv, Fab′. Fc. Fc fragments, rIgG, Fab-H, IgG typeM, Fc5-H etc., including the above but conjugated to drug, polymer, biomolecule, or other moieties.

The biological may be labeled with a visually detectable label such a fluorophore, chromophore, or colorimetric molecules including fluorescein isothiocyanate (FITC), AlexaFluor488. Alternatively, the binding agent is labeled with a chromophore such as 3,3′-diaminobenzidine, or a radionuclide such as lutetium-177. These labels are commercially available, e.g., from ThermoFisher Scientific, and/or Sigma Aldrich (Allentown, Pa.).

The biological may be conjugated to a drug molecule, such as, but not limited to, an anti-neoplastic agent, which may or may not be conjugated to the antibody by way of a cleavable molecular linker. For example, the drug molecule may be vedotin, chemically conjugated with a maleimide attachment group to a cathepsin-cleavable linker, to brentuximab. This antibody-drug conjugate is available, e.g., from Takeda/Seattle Genetics (Deerfield, Ill.).

The biological may be conjugated to biomolecules or polymers which are intended to improve the pharmacokinetic characteristics of the biological (e.g., to improve penetration, increase circulating half-life, reduce antigenicity, and increase distribution into tissues), including but not limited to poly-L-lysine, polyglycine, polylactic acid, polysaccharide, polyethylene glycol. These conjugates are commercially available, e.g., from Creative Biolab (Shirley, N.Y.). The antibody may also be conjugated to nanoparticles which are intended to enhance detection or allow conduction of radiation to specific cells. Nanoparticles include, but are not limited to, fluorescent nanoparticles, ferric/ferrous nanoparticles, magnetic nanoparticles, quantum dots, etc.

The site in the tissue surrogate to which the biological specifically binds may any location or target in the microtissue which can be reached and bound by the biological or binding fragment thereof. For example, the binding site may be a protein receptor, nuclear receptor, membrane receptor, enzyme, biomolecule, structural protein, regulatory protein, cytokine, marker of cellular differentiation (e.g., CD31), glycoprotein, membrane transport protein, extracellular matrix, elastins, collagens, storage protein, kinase, transport protein, ion channel, histone, immunoglobulin, hormone, hormone receptor, lipoprotein, metalloprotein, cellular membrane, phospholipids, glycolipids,

If the antibody used to treat the tissue surrogate is not labeled, the antibody-treated microtissue is then contacted with a binding molecule that specifically recognizes and binds the antibody or binding fragment thereof. Representative binding agents include other antibodies which are antigenic for the unlabeled antibody, and binding fragments thereof. The binding agent, itself, can be labeled with a colorimetric or other molecule that can be detected, as described above. One non-limiting binding agent labeled with a fluorophore is mouse anti-mouse IgG AlexFluor488 conjugate.

The labeled tissue surrogates are then rendered optically transparent using a tissue clearing technique such as Visikol® HISTO-M™, Visikol® HISTO-1™ or Visikol® HISTO-2™. Other useful tissue clearing techniques include, but are not limited to, water, Focusclear™, BABB, 3DISCO, iDISCO, CUBIC, PACT, PARS, CLARITY, Scale, ClearT (Richardson, et al. (2015). Cell, 162(2): 246-257), glycerol ClearT2, uDISCO or SeeDB (Moy et al. (2015). J. Biomed Opt, 20 (9): 095010) fructose.

Imaging is then performed using a confocal microscope (e.g., a CellInsight High Content Confocal CX7-LZR microscope, Thermo Fisher, Waltham, Mass.) such as an upright or inverted confocal with a digital camera. Imaging with confocal microscopy is conducted through moving the microscope objective's focal plane through the z-axis of the fluorescently labeled and cleared tissue surrogate model which generates digital images throughout the z-depth of the model.

The resulting digital image stacks are then processed using a computer program as described below.

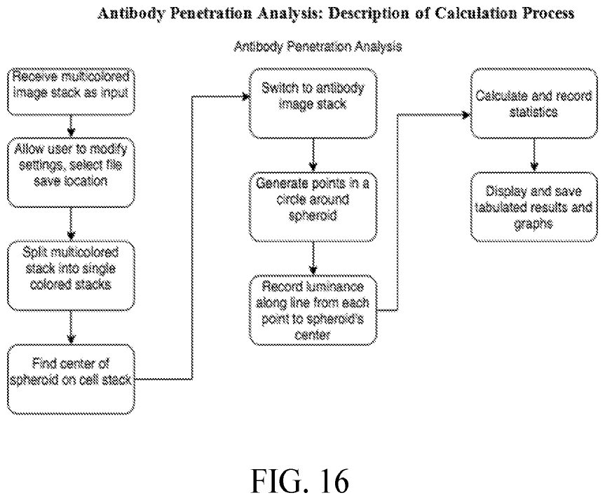

The calculation process is shown in FIG. 16 and uses an Image J and an image J “plugin”. The Image J plugin is a program capable of calculating the penetration of antibodies throughout a tissue surrogate, generates a statistical analysis, and has been created and implemented in the ImageJ Macro language. This program takes in a group of sequential microscope images, referred to as an “image stack,” taken of a tissue surrogate treated with a clearing agent such as Visikol HISTO-M and fluorescent stains. Image sequenced at different Z-axis positions throughout entirety of tissue. Multiple color channels, hereunder referred to as “channels”, each representing a separate fluorophore detected in the image, are taken at each Z-axis position throughout the image stack. Using multiple color channels allows for the independent assessment of multiple biologicals on a single tissue surrogate.

The user is asked to identify the “biological channel” (the channel of the multichannel image stack which represents the biological). The color layer is extracted from digital images. For each image there are typically four to five color channels, all independent “gray” images that can be assembled into a multi-color image. The user is also asked to identify the nuclear channel (the channel of the multichannel image stack which represents the nuclei of the cells in the surrogate), and to choose a file save location. The number of points generated, along which to measure intensity of pixel in the selected antibody channel, may also be altered.

The multichannel image stack is then divided into a series of single-channel image stacks. Each image color channel corresponds to a wavelength of absorption of the stains used. For example, when given a stack of images wherein channel 1 is the nuclear stain channel, and channel 2 is the antibody channel, the program generates one stack for the nuclear channel, and one stack of the antibody channel Each single-colored single-channel image stack represents the different stains on the same imaged tissue surrogate. The program then selects the single-colored image stack that represents the nuclei of the cells in the tissue surrogate.

The program then finds the X and Y center of the tissue surrogate. To accomplish this, the program “collapses” the different z images from the image stack into a single image (known as a “z-projection”) and analyzing the resulting image to detect the tissue surrogate, then it calculates the bounding box and finds the centroid. This is accomplished by applying a thresholding technique to segregate foreground (bright) pixels from background (dark) pixels, and then measuring the boundaries of the foreground object to determine its width, height, and center of mass.

Firstly, a “z-projection” is created. The most common technique for creating a z-projection is by projecting the maximum intensity pixels along the projected perspective rays for each z-plane image of the image stack onto a single image, creating a 2-dimensional representation of the overall z-stack, known as a “maximum intensity z-projection”. Any technique for creating z-projections is amenable to this process, including, but not limited to, maximum z-projection, sum projection (where pixel intensities for each xy pixel are summed across the z axis), average projection (where average pixel intensities for each xy pixel are calculated across z-axis).

Secondly, the flattened image is analyzed for large (500 μm-50000 μm in area) contiguous regions. This is accomplished by “thresholding” the μm² image. The pixel intensity values for the image are analyzed and pixels above the threshold value are included in subsequent analyses, and pixels below that threshold value are ignored for subsequent analyses. This is accomplished by using methods including, but not limited to, the Otsu method, to determine the threshold value. The Otsu method calculates the threshold value by grouping all pixel values in the image into two groups (high or low), then calculating the average pixel value in each group, and finally determining the threshold value by calculating the average of averages for the high and low pixel intensity groups respectively. The threshold value is then applied to the image in order to eliminate pixels below the threshold from subsequent analysis. Thresholding effectively limits the image to the brightest parts of the image, eliminating background pixels from consideration.

After the image threshold is applied, contiguous pixel regions, hereafter referred to as “particles,” in the image exceeding a minimum size (defined between 500 μm²-50000 μm² by the analyst, depending on the size of the tissue surrogate) in the image are detected by iteration through all pixels exceeding the threshold in the image. Contiguous regions are defined as pixels that share at least 1 edge with other pixels. The bounding box and XY center-point is determined for all particles that exceed the minimum size within the image.

The bounding box is determined as the four corner points enclosing the object in a rectangle. The four points are: 1) minimum X, and 2) minimum Y coordinates in the contiguous region, 3) maximum X, and 4) maximum Y coordinates in the contiguous region.

The centroid is determined as the averages of the X and Y coordinates for all pixels contained within the contiguous region. Alternatively, the center-point of the bounding box may be selected as the center instead of the centroid, for may be useful for analysis of particularly uncircular or non-uniform tissue surrogates.

After finding the X and Y center of the tissue surrogate, the program finds the Z center of the tissue surrogate by counting the number of cells in the nuclear channel for each image in the single-colored nucleus image stack. This is accomplished by utilizing the same thresholding and particle analysis as described above, however, using different criteria for minimum size, and instead of processing a flattened z-projection, each slice of the image stack is analyzed. For cell-counting, the minimum size is reduced so that individual cell nuclei are detected (typically 50-500 μm², defined by analyst and input into program depending on cell type). The count of detected particles (nuclei) is used to determine the central Z slice of the stack: the center slice is defined as the slice containing the greatest number of nuclei detected.

Alternatively, the center slice may be determined by evaluation of the total area of pixels exceeding the threshold (defined above) of the tissue surrogate within each z-slice of the image. The slice with the largest area is defined as the center slice. Optionally, the user may manually define the center point.

Once the X, Y, and Z center of the tissue surrogate is determined, the program switches the channel of the image stacks from the nuclear channel to the antibody channel, as defined by the user. The antibody channel stack contains an image of the same spheroid in the same location; however the pixel values represent the color channel used to image the dye which labels the biological, corresponding to wavelength of absorption. Using the same X, Y, and Z coordinates; the program generates a series of points around the outer boundary of the tissue surrogate, equidistant from the center point.

The radius of the tissue surrogate (r) is determined as the half of the maximum dimension (either width or height, whichever is greater) of the bounding box calculated previously. The user inputs the number of points (n) that are desired to be generated around the tissue surrogate. The step-size for the angle between points is calculated as 360°/n, where n is the number of points input by the user, e.g., if user inputs 4 points, each point is separated by 90 degrees. Point coordinates are calculated using the following formulae, increasing θ from 0 to 360 by step-size of 360/n:

(X−X_{center})=r cos(θ)

(Y−Y_{center})=r sin(θ)

For each point around the center, the program records the pixel intensity (0 is totally dark, ranging to a maximum value of (2^{b}−1) for images of b bits, e.g., 255 for 8 bit image), of the image along the line between an outside point and the center point. The intensity for each point along this line is recorded by the program automatically and stored in a file on the computer. This data is hereafter referred to as the “pixel profile curve”.

The program then optionally applies a smoothing function to filter out background noise by calculating the average of consecutive points spanning the pixel profile curve by a step-size defined by the user.

The program calculates the maximal pixel value along each pixel profile curve, by examining each point on the curve and determining the maximum. This value is referred to as the “Vₘₐₓ”

The program calculates the distance from the center along each path to the point at which maximal pixel intensity is found on this path, by iterating through each data point and comparing to the highest pixel value currently found. This value is referred to as the “Dₘₐₓ” value, indicating the distance at which the maximum concentration of antibody was detected.

The program calculates the area under the pixel profile curves by summation of the intensity values of the pixel profile curve plot. The integrated area under the pixel profile curve (AUC) represents the “total” amount of antibody that has penetrated along a specified path.

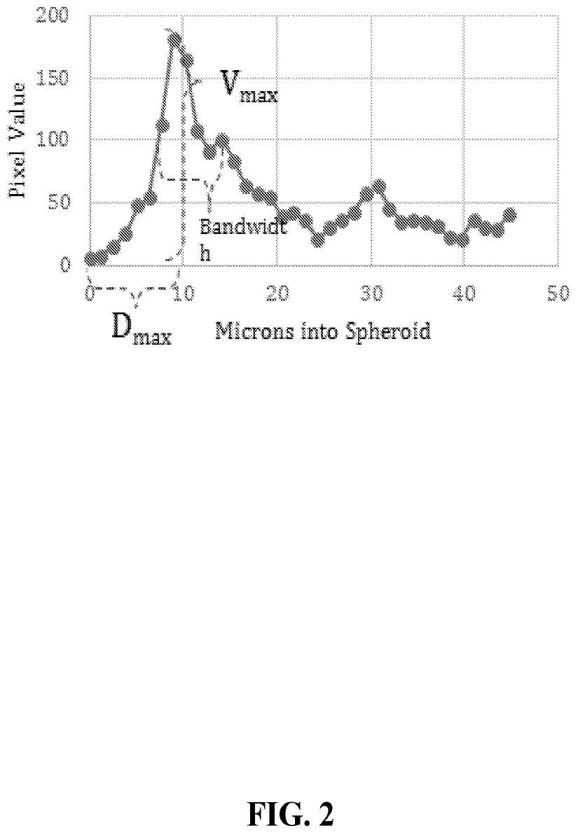

The program calculates the “bandwidth. Bandwidth is the width in microns of the shoulders of the peak at 50% Vₘₐₓ on the profile curve. This value represents the spread of the antibody concentration along a specified path. Vₘₐₓ is a useful value for comparison of the intensity of penetration of the antibody, whereas AUC is the total cumulative sum of the antibody signal along the profile, allowing rank order comparison of the extent of antibody detected within the islet. Pₘₐₓ is the maximum distance into the islet at which antibody is detected. A graphic depiction of these values is shown in FIG. 2.

The program calculates the maximum penetration point defined as the farthest distance from the perimeter of the tissue surrogate where the biological was detected. The program saves this information to disk. Averages of the values are calculated and saved.

The program saves the pixel profile curves, and the various parameters calculated from the pixel profile curves, to disk. Averages of the values are calculated and saved. The program generates plots for each pixel profile curve.

Reference will now be made to specific examples illustrating the disclosure. It is to be understood that the examples are provided to illustrate exemplary embodiments and that no limitation to the scope of the disclosure is intended thereby.

### EXAMPLES

### Example 1

### Extent of Penetration of Anti-O-Integrin Antibody into HCT116 3D Cell Cultures

### Methodology

HCT116 3D cell cultures (InSphero AG, Brunswick, Me.) and A498 3D cell cultures (InSphero AG, Brunswick, Me.) were treated with anti-human β-integrin (mouse IgG) (Fisher Scientific, Waltham, Mass.) at 20 μg/mL for 10 min to 4 hr. The 3D cell cultures were fixed for 10 minutes in neutral buffered formalin, and processed for labeling according to the instructions at protocol.visikol.com. Briefly, the 3D cell cultures were labeled with mouse anti-mouse IgG-AlexaFluor488 conjugate at 1:200 dilution (10 μg/mL) and DAPI by incubation for 1 hour at room temperature protected from light. The 3D cell cultures were washed 5 times for 5 minutes each time with PBS containing 0.2% Triton X-100, and 10 μg/mL heparin. Excess fluid was removed from the 3D cell culture model with a pipette. The 3D cell cultures were then cleared using Visikol® HISTO-M™. Cleared 3D cell cultures were imaged using a CX7-LZR (ThermoFisher, Waltham, Mass.) using confocal mode at a 5-micron z-step size. Z-stacks were further processed using an ImageJ (National Institute of Health, Bethesda, Md.) plugin used to extract pixel values along paths in images.

### Image Processing

Pixel values were extracted along user-defined paths originating from the center point in the 3D cell culture to generate antibody penetration curves. An image of the 3D cell culture from the center z within the stack was used to determine the extent of antibody penetration into the core of the tissue rather than on the surface.

Four parameters were calculated for each path: Bandwidth, Dₘₐₓ , Vₘₐₓ, and AUC (area under the curve). Bandwidth is the width in microns of the shoulders of the peak at 50% Vₘₐₓ on the profile curve. Dₘₐₓ is the distance into the 3D cell culture at which the maximal pixel value (Vₘₐₓ) occurs in the antibody channel image, and AUC is the integrated area under the curve. Dₘₐₓ is a useful numerical comparison of the center of the “band” of antibody as it penetrates the 3D cell culture. Vₘₐₓ is a useful value for comparison of the intensity of penetration of the antibody, whereas AUC is the total cumulative sum of the antibody signal along the profile, allowing rank order comparison of the extent of antibody detected within the 3D cell culture. A graphical depiction of these values is shown in FIG. 2.

### Results

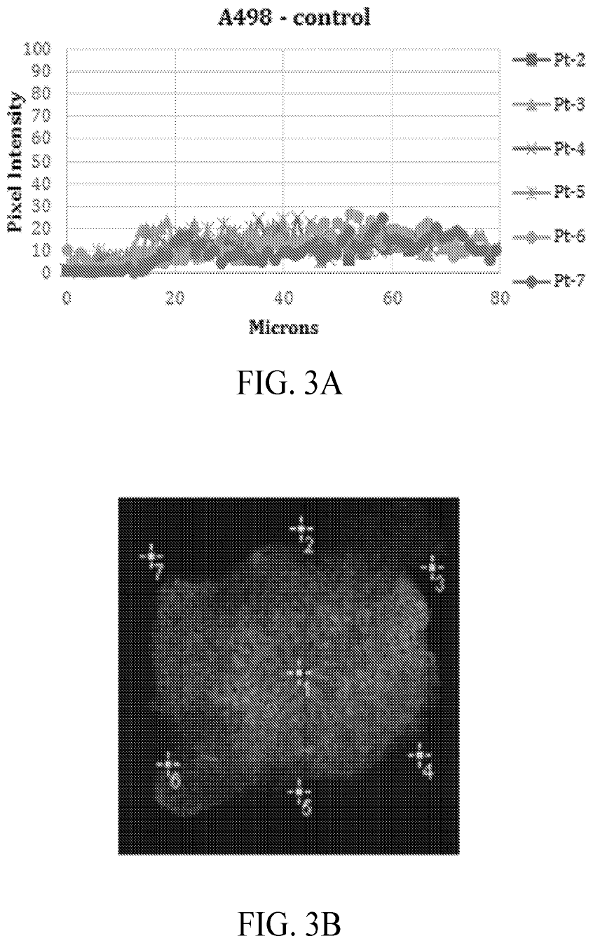

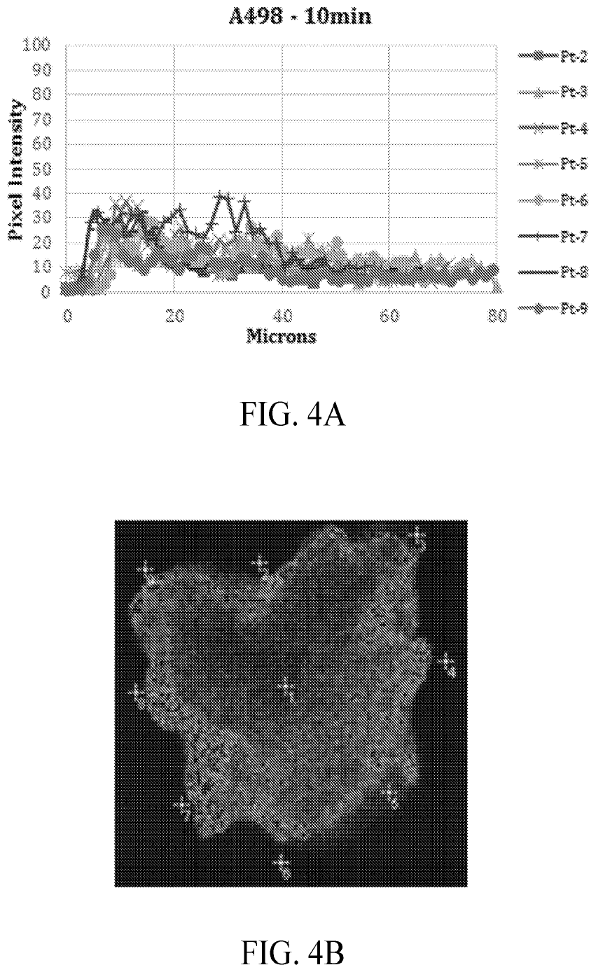

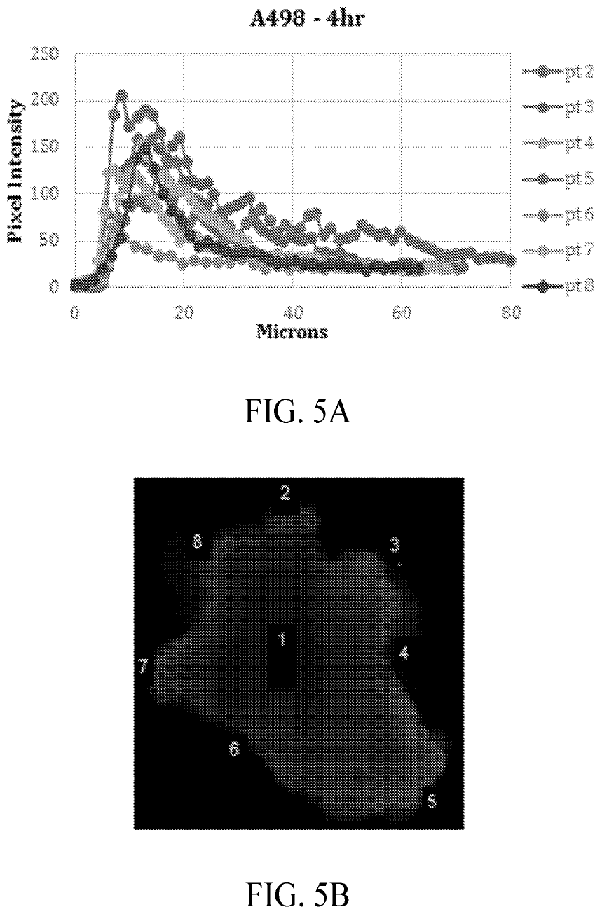

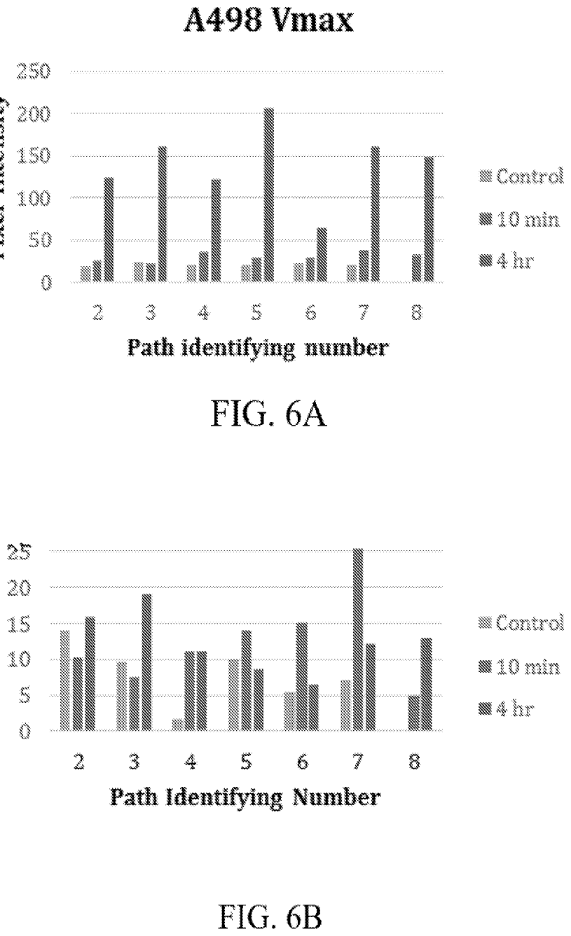

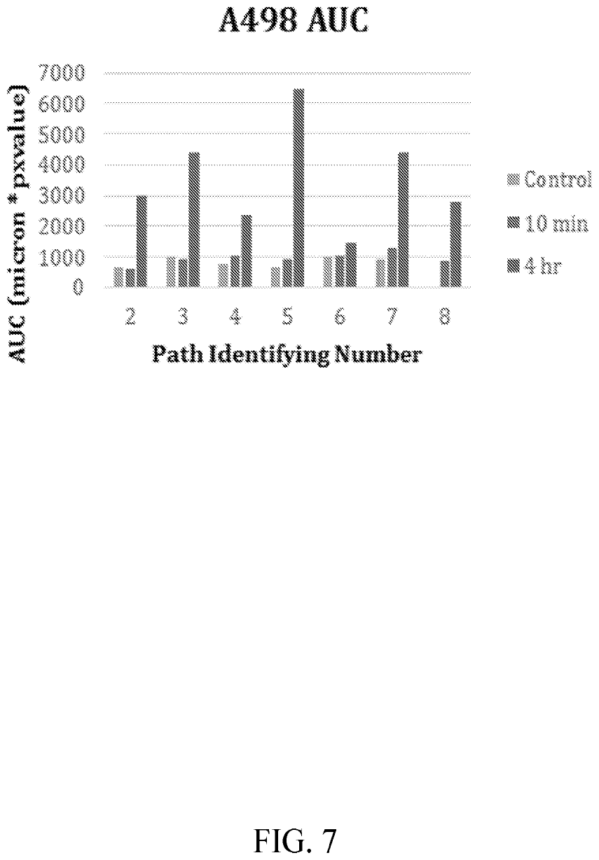

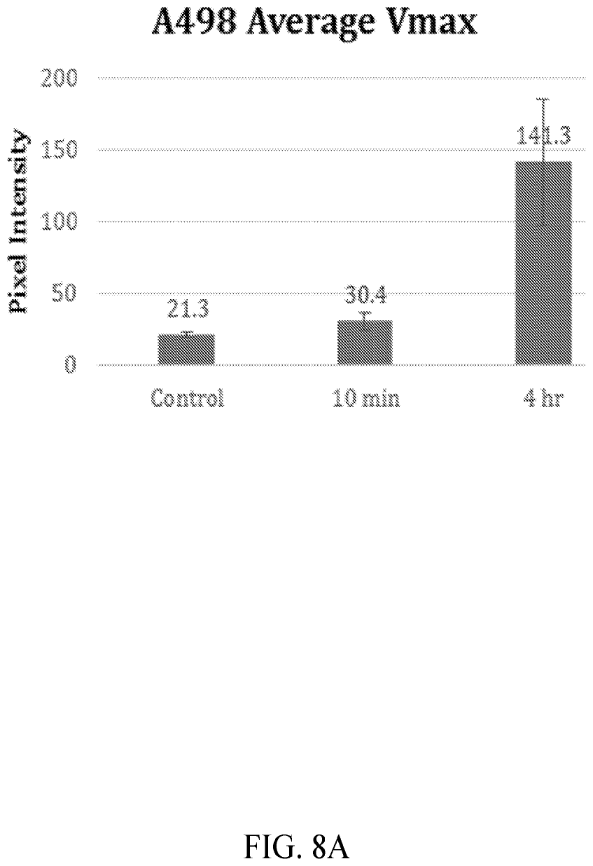

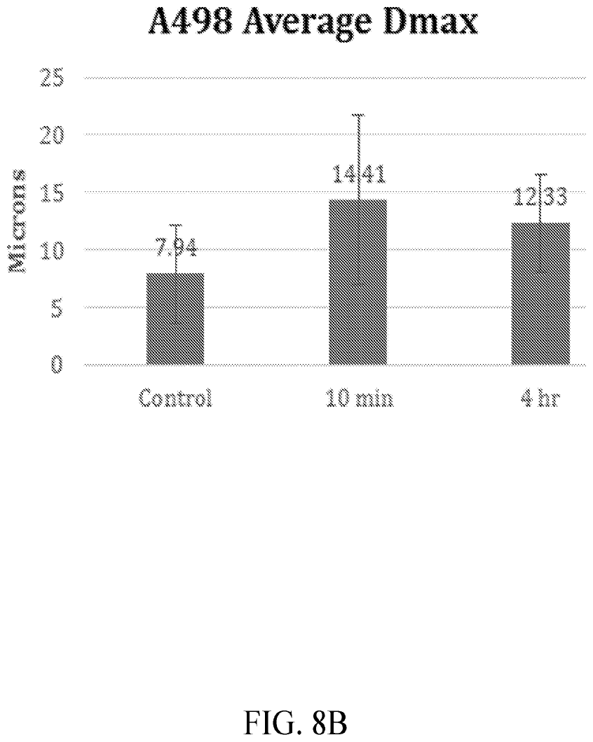

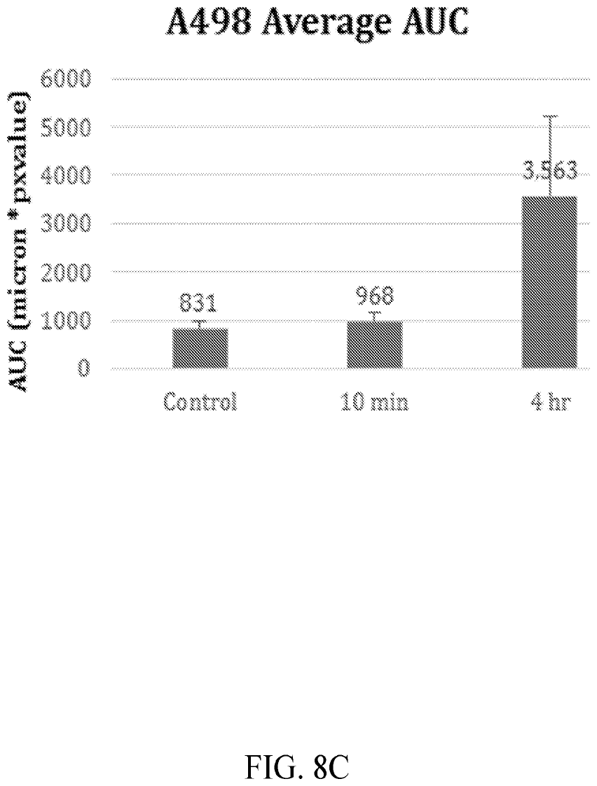

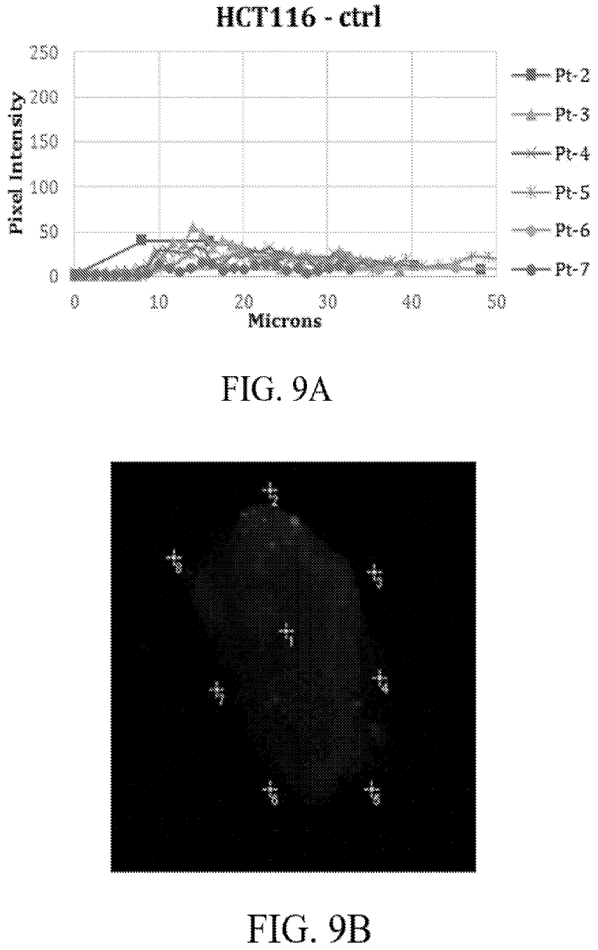

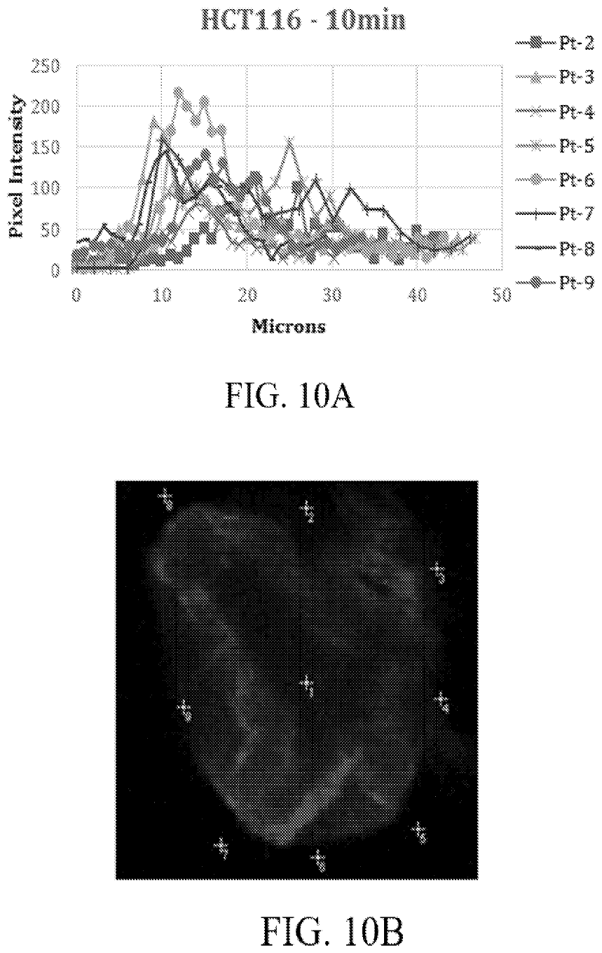

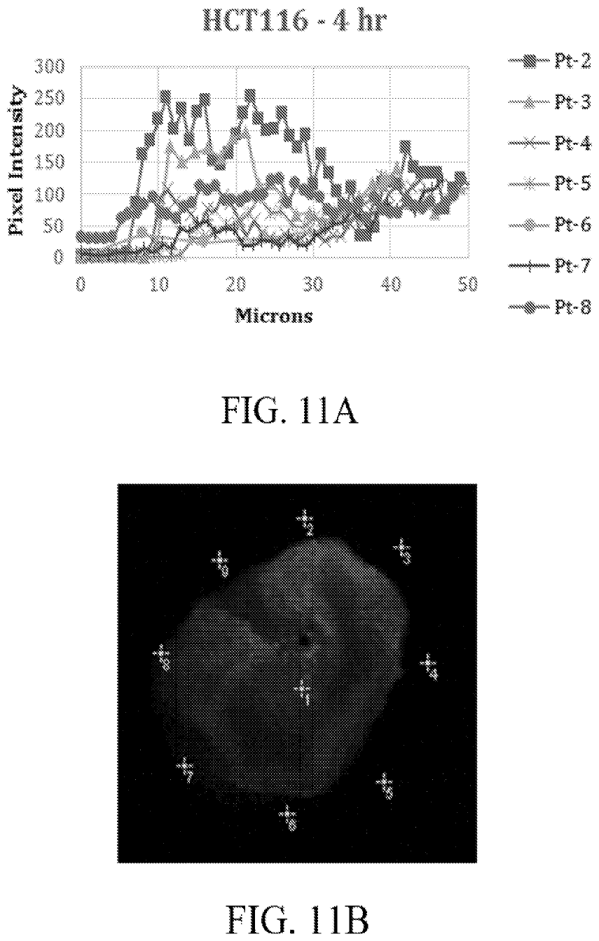

The results of quantification of the extent of antibody penetration into A498 and HCT116 cancer 3D cell cultures obtained from InSphero demonstrate a novel method to evaluate the relative propensity of various therapeutic antibodies to penetrate tumor 3D cell cultures. The quantitative profile curves in FIGS. 3-11 showing the relative concentration of beta-integrin throughout untreated control (FIG. 3A), 10 min (FIG. 4A) incubation, and 4 hr. (FIG. 5A) incubation for A498 and HCT116 3D cell cultures (FIG. 9A, control), FIG. 10A (10 min) FIG. 11A (4 hr.) are shown in FIGS. 3 and 5, respectively, along with reference images depicting the points along which the curves were generated. The profile curves depict pixel intensities as a function of distance from the higher numbered point (2-9) along the straight line connecting this point to point 1. From these curves, the overall extent of antibody penetration and can be visualized after 10 min and 4 hr incubation compared to control tissues.

Table 1 provides bandwidth values (in microns) for each path profile and average of all paths of A498 3D cell culture treated for 10 min and 4 hr with β-integrin antibody.

TABLE 1

| Path ID^{‡} | | 2 | 3 | 4 | 5 | 6 | 7 | 8 | Average ± stdev |
|---|---|---|---|---|---|---|---|---|---|
| 10 | min* | 20.4 | 19.6 | 9.1 | 11.2 | 14.0 | 40.5 | 14.0 | 19.1 ± 11.39 |
| 4 | hr.* | 17.0 | 24.2 | 11.0 | 14.2 | 13.1 | 24.2 | 9.7 | 16.2 ± 5.94 |

^{‡}Path IDs coincide with labels in FIG. 3 where the path ID indicates the starting point in the path, and point “1” the endpoint.
*Bandwidth in microns

Table 2 provides bandwidth values (in microns) for each path profile and average of all paths HCT116 3D cell cultures treated for 10 min and 4 hr with β-integrin antibody.

TABLE 2

| Path ID^{‡} | | 2 | 3 | 4 | 5 | 6 | 7 | 8 | Average ± stdev |
|---|---|---|---|---|---|---|---|---|---|
| 10 | min* | 8.0 | 7.7 | 9.1 | 23.4 | 8.0 | 14.0 | 10.4 | 11.5 ± 5.28 |
| 4 | hr.* | 25.0 | 14.7 | 34.6 | 20.2 | 7.9 | 12.8 | 30.8 | 19.5 ± 9.78 |

^{‡}Path IDs coincide with labels in FIG. 5 where the path ID indicates the starting point in the path, and point “1” the endpoint.
*Bandwidth in microns

Table 3 provides Area Under the Curve (AUC) values (in microns*Intensity) for each path profile and average of all paths of A498 3D cell culture treated for 10 min and 4 hr with β-integrin antibody.

TABLE 3

| Path ID^{‡} | | 2 | 3 | 4 | 5 | 6 | 7 | 8 | Average ± stdev |
|---|---|---|---|---|---|---|---|---|---|
| 10 | min* | 625.5 | 942.8 | 1037.9 | 901.4 | 1028.6 | 1270.8 | 845.0 | 967.8 ± 211.03 |
| 4 | hr* | 3022.1 | 4416.1 | 2366.8 | 6479.9 | 1450.5 | 4416.1 | 2788.5 | 3562.8 ± 1670.8 |

^{‡}Path IDs coincide with labels in FIG. 3 where the path ID indicates the starting point in the path, and point “1” the endpoint.
*Area Under the Curve in microns*Intensity

Table 4 provides Area Under the Curve (AUC) values (in microns*Intensity) for each path profile and average of all paths of HCT116 3D cell culture treated for 10 min and 4 hr with β-integrin antibody.

TABLE 4

| Path ID^{‡} | | 2 | 3 | 4 | 5 | 6 | 7 | 8 | Average ± stdev |
|---|---|---|---|---|---|---|---|---|---|
| 10 | min* | 1667.0 | 2453.9 | 1215.7 | 2563.2 | 2589.9 | 3073.4 | 1840.4 | 2172.9 ± 601.9 |
| 4 | hr* | 7149.2 | 5369.1 | 2280.8 | 1866.3 | 2063.5 | 2017.2 | 4081.4 | 3474.8 ± 1921.35 |

^{‡}Path IDs coincide with labels in FIG. 4 where the path ID indicates the starting point in the path, and point “1” the endpoint.
*Area Under the Curve in microns*Intensity

Table 5 provides Vmax values (in pixel intensity) for each path profile and average of all paths of A498 3D cell culture treated for 10 min and 4 hr with β-integrin antibody.

TABLE 5

| Path ID^{‡} | | 2 | 3 | 4 | 5 | 6 | 7 | 8 | Average ± stdev |
|---|---|---|---|---|---|---|---|---|---|
| 10 | min* | 25.3 | 23.0 | 36.8 | 29.6 | 28.9 | 39.0 | 33.0 | 30.4 ± 6.32 |
| 4 | hr* | 124.5 | 161.3 | 122.1 | 206.7 | 64.3 | 161.3 | 148.6 | 141.3 ± 44.17 |

^{‡}Path IDs coincide with labels in FIG. 2 where the path ID indicates the starting point in the path, and point “1” the endpoint.
*Vmax in pixel intensity (0-255)

Table 6 provides Vmax values (in pixel intensity) for each path profile and average of all paths of HCT116 3D cell culture treated for 10 min and 4 hr with β-integrin antibody.

TABLE 6

| Path ID^{‡} | | 2 | 3 | 4 | 5 | 6 | 7 | 8 | Average ± stdev |
|---|---|---|---|---|---|---|---|---|---|
| 10 | min* | 112.00 | 182.60 | 80.33 | 155.80 | 216.33 | 159.57 | 143.86 | 148.9 ± 41.41 |
| 4 | hr* | 252.51 | 197.47 | 122.00 | 129.07 | 122.00 | 122.00 | 124.48 | 153.9 ± 48.14 |

^{‡}Path IDs coincide with labels in FIG. 4 where the path ID indicates the starting point in the path, and point “1” the endpoint.
*Vmax in pixel intensity (0-255)

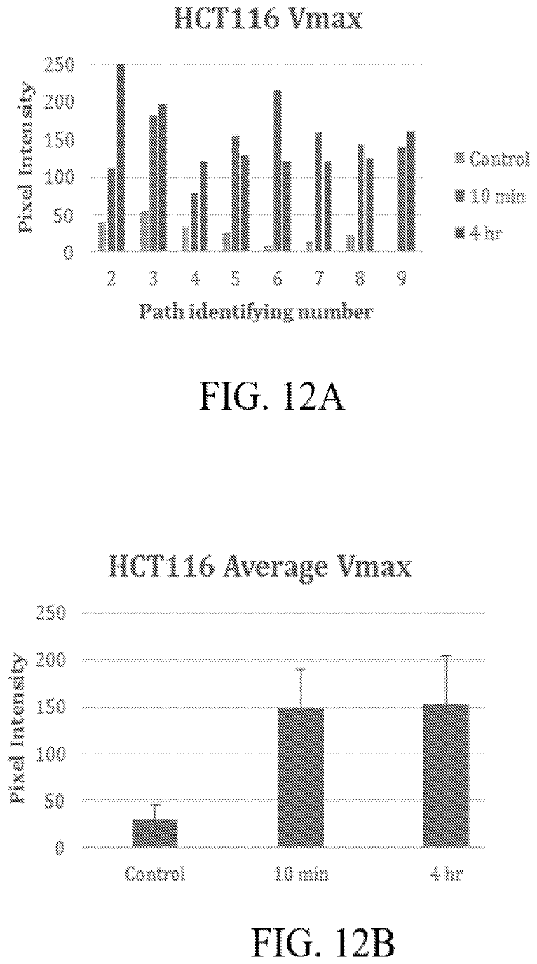

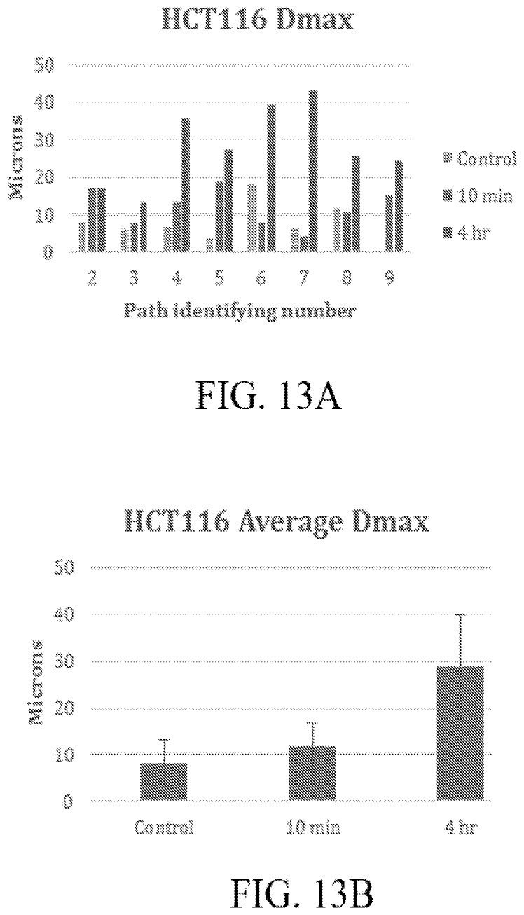

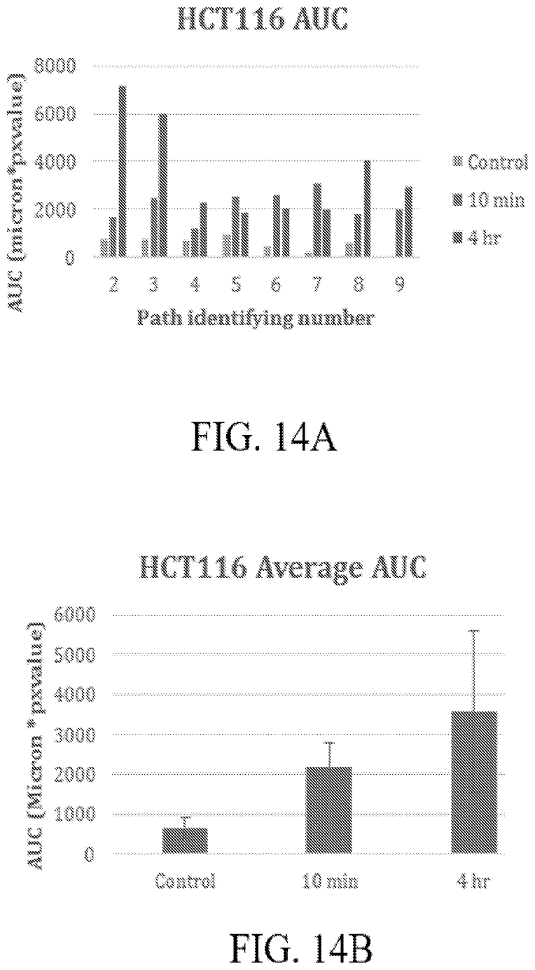

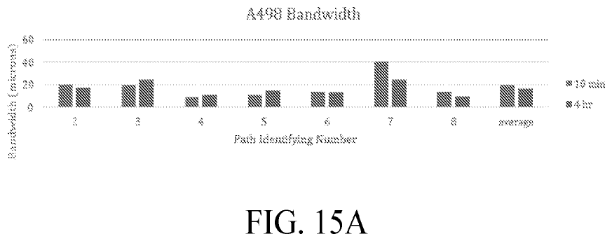

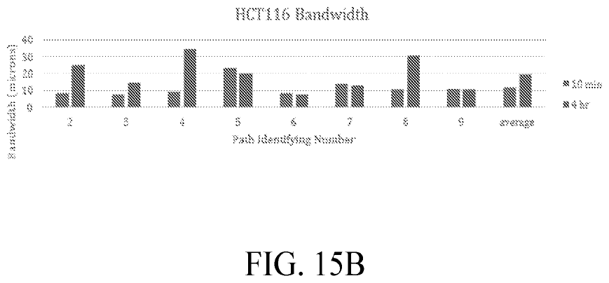

Vₘₐₓ, Dₘₐₓ, and AUC values for each path as well as averages are shown for the 3D cell cultures for A498 and HCT116 are also shown in FIGS. 12-14). Bandwidth values for each path as well as averages±standard deviation for antibody penetration in A498 (FIG. 15A) and HCT116 (FIG. 15B) 3D cell cultures are shown in Table 1 and 2 above, respectively Significant differences in the speed at which antibodies penetrated A498 vs. HCT116 3D cell cultures was detected—antibodies penetrated HCT116 3D cell cultures significantly faster.

### Example 2

### Penetration of β-Integrin Antibody into Cancer 3D Cell Models

Using two cancer 3D cell culture models, MCF-7 and HeLa (Sigma-Aldrich, St. Louis, Mo.), the method described in EXAMPLE 1 is used to determine the ability of mouse anti-human estrogen receptor IgG antibodies (Bio-Rad, Hercules, Calif.) to penetrate these 3D cell cultures. The 3D cell cultures are treated for 10 min or for 4 hr with the antibody solution. The 3D cell cultures are then labeled with a fluorescent binding agent (i.e., secondary antibody) specific for the anti-estrogen receptor antibodies (mouse anti-mouse IgG-AlexaFluor488 conjugate) for 1 hr and then rendered transparent with Visikol HISTO-M. The cleared 3D cell cultures were then imaged using a CX7 LZR high content microscope in confocal mode using a 5 micron z-step size.

The images shows a ring of antibody binding in the MCF-7 cells, and no signal in the HeLa cells, as HeLa cells do not express estrogen receptor, and hence will not be bound by antibody. From these data, an increasingly intense band of antibody in the MCF-7 cells increasing is observed with length of incubation time. Quantitative analysis of this data shows a curve with a peak at about 50 microns into the tissue, and a bandwidth of 70 microns for 10 min incubation. Quantitative analysis of data obtained from four-hour incubation of MCF-7 cells shows a more intense band, with a peak in intensity at 100 microns, and a 150 micron bandwidth, with a Vmax of 180.

### Example 3

### Penetration of Liraglutide into Pancreatic Islet 3D Cell Cultures

### Methodology

Pancreatic islet cell cultures (InSphero AG, Brunswick, Me.) were treated with liraglutide (Novo Nordisk A/S, Denmark) at 20 μg/mL for 10 min to 6 hr. The islets were processed for labeling according to the instructions at protocol.visikol.com. Briefly, the islets were labeled with mouse anti-human GLP1-AlexaFluor488 conjugate at 1:200 dilution (10 μg/mL) and DAPI by incubation for 1 hr. at RT protected from light. The islets were washed 5 times for 5 min each time with PBS containing 0.2% Triton X-100, and 10 μg/mL heparin. Excess fluid was removed from the islet model with a pipette. The islets were then cleared using Visikol® HISTO-M™. Cleared islets were imaged using a CX7-LZR (ThermoFisher, Waltham, Mass.) using confocal mode at a 5-micron z-step size. Z-stacks were further processed using an ImageJ (National Institute of Health, Bethesda, Md.) plugin used to extract pixel values along paths in images.

### Image Processing

Pixel values were extracted along user-defined paths originating from the center point in the islet to generate biological-drug penetration curves. An image of the islet from the center z within the stack was used to determine the extent of biological penetration into the core of the tissue rather than on the surface.

Five parameters were calculated for each path: Bandwidth, Dₘₐₓ, Vₘₐₓ, AUC (area under the curve), and maximal penetration Pₘₐₓ.

### Results

The results of quantification of the extent of liraglutide penetration into pancreatic islets obtained from InSphero demonstrate a novel method to evaluate the relative propensity of various therapeutic biologicals to penetrate pancreatic islets. The profile curves depict pixel intensities as a function of distance from the higher numbered point (2-9) along the straight line connecting this point to point 1. From these curves, the overall extent of antibody penetration is visualized after 10 min, 4 hr, and 6 hr of incubation compared to control tissues. It was shown that liraglutide concentration within islets increases dramatically between 10 min and 4 hr incubation, but only increases slightly after 6 hr incubation. The Pmax value is nearly identical for 4 hr. and 6 hr incubation (56 and 61 microns respectively), but were nearly 10× the value measured for 10 min incubation (6.1 microns). Furthermore, the AUC increased substantially in the 4 hr and 6 hr incubations compared to the 10 min incubation.

### Example 4

### Extent of Penetration of Viral Vector into HepG2 3D Cell Cultures

### Methodology

3D cell cultures comprised of HepG2 cells were generated over the course of 4 days using U-bottom, ultra-low attachment plates (Corning, Corning, N.Y.). HepG2 3D cell cultures were treated with GFP viral vector at 10 ng/mL for 10 min to 6 hr. The HepG2 3D cell cultures were processed for labeling according to the instructions at protocol.visikol.com. Briefly, the spheroids were labeled with mouse anti-GFP-AlexaFluor488 conjugate at 1:200 dilution (10 μg/mL) and DAPI by incubation for 1 hr at RT protected from light. The spheroids were washed 5 times for 5 min each time with PBS containing 0.2% Triton X-100, and 10 μg/mL heparin. Excess fluid was removed from the islet model with a pipette. The spheroids were then cleared using Visikol® HISTO-M™. Cleared spheroids were imaged using a CX7-LZR (ThermoFisher, Waltham, Mass.) using confocal mode at a 5-micron z-step size. Z-stacks were further processed using an ImageJ (National Institute of Health, Bethesda, Md.) plugin used to extract pixel values along paths in images.

### Image Processing

Pixel values were extracted along user-defined paths originating from the center point in the spheroid to generate biological-drug penetration curves. An image of the spheroid from the center z within the stack was used to determine the extent of biological penetration into the core of the tissue rather than on the surface.

Five parameters were calculated for each path: Bandwidth, Dₘₐₓ, Vₘₐₓ, AUC, and Pₘₐₓ.

### Results

The results of quantification of the extent of viral vector penetration into HepG2 spheroids demonstrate a novel method to evaluate the relative propensity of viral vectors to penetrate into cancer tumors. The profile curves depict pixel intensities as a function of distance from the higher numbered point (2-24) along the straight line connecting this point to point 1. From these curves, the overall extent of viral vector penetration is visualized after 10 min and 4 hr incubation compared to control tissues. From these results, it can be seen that the viral vector penetrates only slightly into tissues, even at the maximal incubation time (4 hr). The bandwidth value is only 10 microns, and the Dₘₐₓ is 8 microns, which indicates that there is a narrow band near the surface wherein the viral vector penetrated, yet did not go past the first layer of cells.

### Example 5

### Penetration of Herceptin into 3D-Printed Breast Cancer Model

### Methodology

3D cell cultures comprised of HER2-positive MDA-MB-453 cells (American Type Culture Collection, Manassas, Va.) were 3D-printed. 3D-printed cell cultures were treated with Herceptin (Genentech, South San Francisco, Calif.) at 10 mg/mL for 10 min and 3 hr. The 3D-printed cell cultures were processed for labeling according to the instructions at protocol.visikol.com. Briefly, the 3D-printed cell cultures were labeled with mouse anti-human IgG-AlexaFluor488 conjugate at 1:200 dilution (10 μg/mL) and DAPI by incubation for 1 hr at RT protected from light. The 3D-printed cell cultures were washed 5 times for 5 min each time with PBS containing 0.2% Triton X-100, and 10 μg/mL heparin. Excess fluid was removed from the islet model with a pipette. The 3D-printed cell cultures were then cleared using Visikol® HISTO-M™. Cleared 3D-printed cell cultures were imaged using a CX7-LZR (ThermoFisher, Waltham, Mass.) using confocal mode at a 5-micron z-step size. Z-stacks were further processed using an ImageJ (National Institute of Health, Bethesda, Md.) plugin used to extract pixel values along paths in images.

### Image Processing

Pixel values were extracted along automatically generated paths originating from the center point in the 3D-printed cell cultures to generate biological-drug penetration curves. An image of the 3D-printed cell cultures from the center z within the stack was used to determine the extent of biological penetration into the core of the tissue rather than on the surface.

### Results

Five parameters were calculated for each path: Bandwidth, Dₘₐₓ, Vₘₐₓ, and Pₘₐₓ. A ring of labeling is observed at a distance of 100 microns into the cell model, which shows a corresponding peak in the profile curve at 100 microns, with a 75 micron bandwidth, and an intensity (Vmax) of 220.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

We claim:

1. A method of determining the depth a biological can penetrate into a tissue surrogate, comprising:
treating the tissue surrogate with the biological, or binding fragment thereof, which binds specifically to a predetermined target in and/or on the tissue surrogate;
contacting the biological-treated tissue surrogate with a labeled binding agent specific for the biological or fragment thereof;
imaging the bound label in the microtissue with a 3-D optical microscope and generating an image stack;
calculating the amount and location of biological penetration into the tissue surrogate by:
reading the image stack into memory of a digital image processor; and
processing the image within the digital image processor and the memory, by:
separating the image stack into substacks by wavelength;
locating the tissue surrogate in the image stack;
identifying a center plane of a tissue surrogate in the image stack;
identifying a center of the tissue surrogate; and
measuring an intensity of the bound label at a plurality of distances from the center of the tissue surrogate; and
performing a statistical analysis of the measured intensity of the bound label to determine a measure of the depth a biological can penetrate into the tissue surrogate.

2. The method of claim 1, wherein the intensity of the bound label is measured by measuring bandwidth.

3. The method of claim 1, wherein the tissue surrogate is a portion of a cultured tissue.

4. The method of claim 1, wherein the clearing agent comprises HISTO-M water, Focusclear, BABB, 3DISCO, iDISCO, CUBIC, PACT, PARS, CLARITY, Scale, ClearT, glycerol ClearT2, uDISCO or SeeDB.

5. The method of claim 1, wherein the 3D microscope is a confocal, two-photon, or light-sheet microscope.

6. The method of claim 1, wherein the biological is an antibody or binding fragment thereof.

7. The method of claim 6, wherein the antibody is a monoclonal antibody, antibody mimetic, camelid, humanized antibody, chimeric antibody, or binding fragment thereof.

8. The method of claim 6, wherein the binding fragment of the antibody is an F(ab), Fab′, F(ab′)2, Fv, sFv, r IgG, Fab-H, IgG typeM, Fc5-H, or Fc.

9. The method of claim 1, wherein the binding agent is a receptor, a protein that binds to a receptor on/in the tissue surrogate, an antibody, or binding fragment thereof.

10. The method of claim 1, wherein the binding agent is labeled.

11. The method of claim 10, wherein the label is a fluorescent or colorimetric molecule.

12. The method of claim 11, wherein the location and concentration of biological that has bound to the tissue surrogate is determined by measuring the intensity of the fluorescent label bound thereto.

13. The method of claim 12, wherein fluorescence intensities is measured across multiple arbitrary linear paths within a z-slice.

14. The method of claim 13, wherein the fluorescent intensity is calculated with a modified image J program.

15. The method of claim 1, wherein the tissue surrogate is fixed with a chemical fixative before it is treated with the biological.

16. A method of determining the depth a biological can penetrate into a tissue surrogate, comprising:
treating the tissue surrogate with a labeled biological, or binding fragment thereof, which binds specifically to a predetermined target in and/or on the tissue surrogate;
imaging the bound label in the tissue surrogate with a 3-D optical microscope and generating an image stack;
calculating the amount and location of biological penetration into the tissue surrogate by:
reading the image stack into memory of a digital image processor; and,
processing the image within the digital image processor and the memory, by:
separating the image stack into substacks by wavelength;
locating the tissue surrogate in the image stack;
identifying a center plane of the tissue surrogate in the image stack;
identifying a center of the tissue surrogate;
measuring an intensity of the bound label at a plurality of distances from the center of the tissue surrogate; and
performing statistical analysis of the measured intensity of the bound label to determine a measurement of the depth a biological can penetrate into the tissue surrogate.

17. The method of claim 16, wherein the clearing agent comprises HistoM, water, Focusclear, BABB, 3DISCO, iDISCO, CUBIC, PACT, PARS, CLARITY, Scale, ClearT, glycerol ClearT2, uDISCO or SeeDB.

18. The method of claim 16, wherein the 3Dmicroscope is a confocal, two-photon, or light-sheet microscope.

19. The method of claim 16, wherein the biological is an antibody, or binding fragment thereof.

20. The method of claim 19, wherein the antibody is a monoclonal antibody, antibody mimetic, camelid, humanized antibody, chimeric antibody, or binding fragment thereof.

21. The method of claim 19, wherein the binding fragment of the antibody is an F(ab), Fab′, F(ab′)2, Fv, sFv, r IgG, Fab-H, IgG typeM, Fc5-H, or Fc.

22. The method of claim 16, wherein the label is a fluorescent or colorimetric molecule.

23. The method of claim 22, wherein the location and concentration of biological that has bound to the microtissue is determined by measuring the intensity of the fluorescent label bound thereto.

24. The method of claim 22, wherein fluorescence intensities is measured across multiple arbitrary linear paths within a z-slice.

25. The method of claim 24, wherein the fluorescent intensity is calculated with a modified image J program.

26. The method of claim 16, wherein the tissue surrogate is fixed with a chemical fixative before it is treated with the biological.
